(19) United States
(12) Patent Application Publication
(10) Pub. No.: US 2016/0114058 A1
(43) Pub. Date: Apr. 28, 2016

(54) POLYMER MICELLE PHARMACEUTICAL COMPOSITION

(71) Applicant: NANOCARRIER CO., LTD., Kashiwa-shi, (JP)

(72) Inventors: Yasuki KATO, Kashiwa-shi (JP); Mitsunori HARADA, Kashiwa-shi (JP); Ryosuke TANAKA, Kashiwa-shi (JP)

(21) Appl. No.: 14/891,561

(22) PCT Filed: May 16, 2014
(86) PCT No.: PCT/JP2014/063020

§ 371 Date: Nov. 16, 2015

(30) Foreign Application Priority Data
May 17, 2013 (JP) 2013-105465

(51) Int. Cl. A61K 47/48; A61K 47/10; A61K 9/107; A61K 31/337

(52) U.S. Cl. CPC A61K 47/48692; A61K 31/337; A61K 47/10; A61K 47/48315; A61K 9/107

(57) ABSTRACT

A polymer micelle pharmaceutical composition is provided and includes: a block copolymer unit α having a hydrophilic polymer chain segment and a hydrophobic polymer chain segment; and a block copolymer unit β having a hydrophilic polymer chain segment and a hydrophobic polymer chain segment, wherein: the block copolymer unit α and the block copolymer unit β are radially arranged in the state in which the hydrophilic polymer chain segments are directed outward and the hydrophobic polymer chain segments are directed inward; and the hydrophobic polymer chain segment of the block copolymer unit α is constituted of repeating units having side chains, at least one of the side chains having a hydrophilic group.

### TECHNICAL FIELD

The present invention relates to a polymer micelle pharmaceutical composition.

### BACKGROUND ART

Various kinds of drug delivery systems (DDS) have been previously developed. For example, polymer micelle formulations utilizing a block copolymer having a hydrophilic segment and a hydrophobic segment have been disclosed, e.g., in WO 2009/142326 A1 and WO 2010/013836 A1. Such prior polymer micelle formulation technology has significantly contributed to enhancing drug efficacy while reducing side effects of drugs, but there is still room for further improvement in order to achieve higher efficacy with fewer or less severe side effects.

### SUMMARY OF THE INVENTION

An object of the present teachings is to provide a polymer micelle pharmaceutical composition having reduced side effects while exhibiting a high pharmacological effect.

In the field of the prior polymer micelle technologies, which have a goal of achieving longer-term maintenance of the micelle particle structure in blood, there was a prevailing belief that setting the degree of hydrophobicity of the hydrophobic segment moiety of the block copolymer as high as possible would achieve stronger hydrophobic interactions between block copolymers. For example, there has been a technological inclination to intentionally design, as a micelle constituent material, a block copolymer so that its polyamino acid segment side chains are completely covered with hydrophobic structures such as benzyl groups (i.e. benzyl group introduction ratio: 100%). This technical inclination was particularly prominent when a block copolymer, which does not have a drug bound thereto, was selected as a micelle constituent material.

However, to the contrary, the present inventors have found that, when a block copolymer (e.g., a third polymer), which weakens the contribution based on hydrophobic interactions to the strong maintenance of the micelle particle structure, is utilized as one component of the micelle constituent materials, the pharmacological effect of the drug to be delivered can be greatly enhanced while the drug side effect-suppressing effect of the polymer micelle technology is exhibited.

That is, according to one aspect of the present teachings, a polymer micelle pharmaceutical composition is provided and preferably includes:

a block copolymer unit α having a hydrophilic polymer chain segment and a hydrophobic polymer chain segment; and

a block copolymer unit β having a hydrophilic polymer chain segment and a hydrophobic polymer chain segment,

wherein:

the block copolymer unit α and the block copolymer unit β are arranged in the state in which the hydrophilic polymer chain segments are directed radially outward and the hydrophobic polymer chain segments are directed radially inward; and

the hydrophobic polymer chain segment of the block copolymer unit α is constituted of repeating units having side chains, at least one of the side chains having a hydrophilic group.

According to the present teachings, a polymer micelle pharmaceutical composition is provided, in which both of the enhancement of the pharmacological effect of the drug and the suppression of side effects become more remarkable.

### BRIEF DESCRIPTION OF THE DRAWINGS

FIG. 1 is a schematic diagram of a polymer micelle pharmaceutical composition according to one embodiment of the present teachings.

FIG. 2 is a graph that shows the results of a cellular uptake test for the micelle formulations of Test Examples 1 to 6.

### DETAILED DESCRIPTION

1. Polymer Micelle Pharmaceutical Composition

A polymer micelle pharmaceutical composition according to one embodiment of the present teachings is described in detail below. The polymer micelle pharmaceutical composition contains one or more block copolymer units α (polymer unit α) and one or more block copolymer units β (polymer unit β). Each polymer units α has a hydrophilic polymer chain segment 11 and a hydrophobic polymer chain segment 12. Each polymer units β has a hydrophilic polymer chain segment 21 and a hydrophobic polymer chain segment 22. The polymer unit(s) α and the polymer unit(s) β are arranged in the state in which the hydrophobic polymer chain segments 12 and 22 are each directed radially inward, and the hydrophilic polymer chain segments 11 and 21 are each directed radially outward. The hydrophobic polymer chain segment 12 of the polymer unit α is constituted of repeating units having side chains. At least one of the side chains has a hydrophilic group. The hydrophilic polymer chain segment of each of the polymer unit α and the polymer unit β is typically a polyethylene glycol chain. The hydrophobic polymer chain segment of each of the polymer unit α and the polymer unit β is typically a polyamino acid chain. It should be noted that the block copolymer unit α is hereinafter sometimes referred to as “backbone polymer unit α” and the block copolymer unit β is hereinafter sometimes referred to as “backbone polymer unit β.” Further, any appropriate hydrophilic group may be utilized as the hydrophilic group herein. Examples of appropriate hydrophilic groups include a hydroxyl group, a carboxyl group, and an amino group.

To satisfy the condition that the block copolymer units are radially arranged according to the present teachings, it is only required that the block copolymer units are in an aggregated state in which the hydrophobic polymer chain segments are directed radially inward and the hydrophilic polymer chain segments are directed radially outward. The micelle may have a slightly collapsed radially arranged structure, in which points of origin for the arrangement of the respective block copolymer units are not focused on a single point. The polymer micelle pharmaceutical composition may be such that the polymer aggregate of the block copolymer units is in a dry state.

According to the above polymer micelle pharmaceutical composition, the therapeutic index can be greatly improved as compared to prior polymer micelle formulations. Although the mechanism by which such effect is obtained is still unclear, it is presumed to be as follows. First, the polymer micelle has the property of being easily retained in the surroundings of a target cell by virtue of the EPR effect due to its particle size. In addition, it is considered that, in the polymer micelle pharmaceutical composition according to the present teachings, the backbone polymer unit α is likely to be detached early from the micelle by virtue of the degree of hydrophilicity of its hydrophobic segment moiety. When the unit α is released from the micelle, while the micelle is retained in proximity of the target cell, the cell membrane of the target cell present in the vicinity of the retained micelle is easily stimulated by the hydrophobic groups of the hydrophobic segment moiety of the unit α, resulting in activation of the endocytosis process of the cell. As a result, it is considered that the delivery properties of the polymer micelle pharmaceutical composition including the drug to be delivered to the target cell are enhanced, and the pharmacological effect of the drug is more efficiently exhibited.

The polymer micelle pharmaceutical composition 100 may further include one or more block copolymer units γ (hereinafter sometimes referred to as “backbone polymer unit γ”) having a hydrophilic polymer chain segment 31 with a target binding site 33 bound thereto, and a hydrophobic polymer chain segment 32. The backbone polymer unit(s) γ may be arranged together with the backbone polymer units α and β in the state in which the hydrophilic polymer chain segment 31 is directed radially outward and the hydrophobic polymer chain segment 32 is directed radially inward.

According to the present teachings, the term “target binding site” means a site having a biological recognition function, which can specifically bind to a substance originating from organisms and viruses and can form a biological binding pair with the substance. Examples of substances originating from organisms and viruses may include molecules present in biological cells, bacteria, fungi, and viruses. Examples of the biological cells may include: tumor cells, neovascular cells, and surrounding cells thereof; immunocompetent cells (such as B cells); inflammatory cells (such as leukocytes); vascular endothelial cells; and cells constituting various organs. The target binding site may be formed such that it contains, as at least part of its structure, a compound, such as a protein, a peptide, or a sugar chain, which is capable of forming a binding pair with such substance.

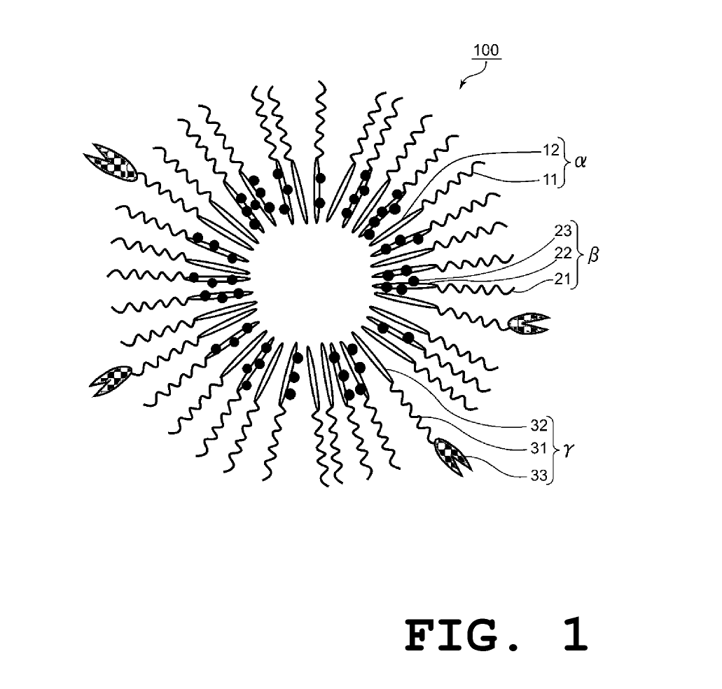

With regard to the incorporation of the drug into the polymer micelle pharmaceutical composition, for example, it is possible that the drug is encapsulated within the micelle, and for example, it is possible that the drug is carried on the surface of the micelle. Although each drug molecule is typically bound to a block copolymer unit, it need not be bound to a block copolymer unit as long as the object of the present teachings is not adversely affected. In case the drug is encapsulated within the micelle, the site(s) at which the drug(s) is (are) bound to the block copolymer unit may be a side chain and/or an inwardly projecting end of the hydrophobic polymer chain segment; in case the drug is carried on the surface of the micelle, it may be bound to an outwardly projecting end of the hydrophilic polymer chain segment. In case the drug is encapsulated in the micelle, the drug(s) is (are) typically bound to (a) side chain(s) of the repeating unit of the hydrophobic polymer chain segment. In the example illustrated in FIG. 1, the drugs 23 are bound to the hydrophobic polymer chain segments 22 of the backbone polymer units β. Specific examples of the drug and the like are described below. It should be noted that the polymer micelle pharmaceutical composition according to the present teachings typically contains the drug, but polymer micelle pharmaceutical compositions that contain no drug are not excluded from the present teachings. For example, the polymer micelle pharmaceutical composition according to the present teachings may be contain the target binding site(s) while containing no drug.

Furthermore, the polymer micelle pharmaceutical composition may contain no target binding site. However, in order to achieve the effect(s) of the present teachings at a higher level, the polymer micelle pharmaceutical composition preferably contains the target binding site(s). This is because a polymer micelle retained in proximity of the target cell easily accumulates more locally toward the target cell by virtue of the EPR effect. As demonstrated in the below-described Test Examples as well, the polymer micelle pharmaceutical composition according to the present teachings enables the delivery of the drug to the target cell to an extent beyond the original (innate) ability of a compound serving as the target binding site to deliver the drug to the target cell (such as the ability of an antibody to be internalized in the cell membrane). FIG. 1 is an illustration of an aspect in which target binding sites 33 are bound to the surface of the polymer micelle pharmaceutical composition 100. More specifically, the target binding sites 33 are bound to the hydrophilic polymer chain segments 31 of the backbone polymer units γ. It should be noted that the polymer micelle pharmaceutical composition according to the present teachings does not exclude embodiments in which a target binding site is also bound to a block copolymer unit other than the backbone polymer unit γ.

The content of the backbone polymer unit α in the polymer micelle pharmaceutical composition is, for example, 15 wt % or more, for example, 20 wt % or more, and for example, 35 wt % or more. As demonstrated in below-described Example and Test Examples, when the polymer micelle pharmaceutical composition contains a predetermined amount or more of the unit α, the delivery properties of the polymer micelle pharmaceutical composition to the target cell can be more certainly enhanced, and the pharmacological effect of the drug to be delivered can be greatly augmented. The upper limit of the content of the unit α is not particularly limited, but from the viewpoint of preventing excessive collapsing of the polymer micelle pharmaceutical composition, more specifically, from the viewpoint of securing the sustainability of the micelle structure until delivery to the vicinity of the target cell in blood while exhibiting the side effect-preventing action of the polymer micelle pharmaceutical composition, the upper limit may be set to, for example, 80 wt %, for example, 60 wt %, for example, 50 wt %, and for example, 45 wt %.

The content of the backbone polymer unit β in the polymer micelle pharmaceutical composition is, for example, 80 wt % or less, for example, 70 wt % or less, and for example, 60 wt % or less. The lower limit of the content of the unit β is not particularly limited, but from the viewpoint of preventing an excessive decrease in the loading ratio of the drug to be delivered in the polymer micelle pharmaceutical composition, the lower limit may be set to, for example, 15 wt %, for example, 25 wt %, and for example, 40 mass %. When the backbone polymer unit γ is contained in the polymer micelle pharmaceutical composition, its content may be set as follows: (1) the lower limit is, for example, 1 wt %, for example, 3 wt %, for example, 5 wt %, and for example, 10 wt %; and (2) the upper limit is, for example, 20 wt %, and for example, 15 wt %.

In the polymer micelle pharmaceutical composition, the backbone polymer unit α and the backbone polymer unit β may be present at any appropriate ratio, and may be present at a molar ratio (former:latter) in, for example, the range of from 1:20 to 20:1, preferably the range of from 1:10 to 10:1, more preferably the range of from 1:5 to 5:1, still more preferably the range of from 1:2 to 2:1.

The backbone polymer unit α and the backbone polymer unit β, and other backbone polymer units (such as the backbone polymer unit γ, and (a) backbone polymer unit(s) different from any of the backbone polymer units α, β, and γ) may be present at any appropriate ratio, and may be present at a molar ratio (total molar amount of backbone polymer units α and β:total molar amount of other backbone polymer units) in, for example, the range of from 100:0 to 100:300, preferably the range of from 100:1 to 100:100, more preferably the range of from 100:2 to 100:50.

The polymer micelle pharmaceutical composition 100 may include two or more kinds of block copolymer units αs each of the above-mentioned block copolymer units.

2. Backbone Polymer Unit α

As was noted above, the backbone polymer unit α contains the hydrophilic polymer chain segment and the hydrophobic polymer chain segment.

The hydrophobic polymer chain segment of the backbone polymer unit α is constituted of repeating units having side chains. At least one of the side chains has a hydrophilic group. In the side chains of the hydrophobic polymer chain segment of the backbone polymer unit α, the percentage of side chains having a hydrophilic group is, for example, 20% or more, and for example, 35% or more. As demonstrated in the below-described Example and Test Examples, when the percentage of the side chains having a hydrophilic group is controlled (set) to a predetermined amount or more, the delivery properties of the polymer micelle pharmaceutical composition to the target cell can be more certainly enhanced, and the pharmacological effect of the drug to be delivered can be greatly augmented. The upper limit of the percentage of the side chains having a hydrophilic group is not particularly limited, but from the viewpoint of preventing excessive collapsing of the polymer micelle pharmaceutical composition, the upper limit may be set to, for example, 80%, for example, 60%, and for example, 50%.

Although the mechanism by which the delivery properties of the polymer micelle pharmaceutical composition to the target cell can be more certainly enhanced through the control of the content of the backbone polymer unit α in the polymer micelle pharmaceutical composition and the ratio of the side chains each having a hydrophilic group in the side chains of the hydrophobic polymer chain segment to the above-mentioned ranges is still unclear, it is presumed, for example, to be as follows. As described above, it is considered that the backbone polymer unit α is likely to be detached early from the micelle by virtue of the degree of hydrophilicity of its hydrophobic segment moiety. It is considered that when the percentage of the side chains having a hydrophilic group in the side chains of the hydrophobic polymer chain segment of the unit α is controlled (set) to the above-mentioned range, the early detachment of individual units α from the micelle can be more certainly induced. In addition, it is considered that when the content of the unit α in the micelle is controlled to the above-mentioned range, such an amount of the unit α as to be able to activate the endocytosis process of the target cell at a high level can be more certainly (reliably) detached early from the micelle. As a result, the delivery properties of the polymer micelle pharmaceutical composition, which includes the drug to be delivered to the target cell, are enhanced. It should be noted that even if a person skilled in the art of polymer micelles were to have previously considered to add a block copolymer, which does not have a drug bound thereto, as a micelle constituent material in order to improve the function of the micelle, he or she would generally have been inclined to keep its addition ratio as low as 10 mass % or less from the viewpoint of maintaining the drug loading ratio per micelle high. In this connection, as demonstrated in the below-described Test Examples, when the backbone polymer unit α is added at 10 mass %, the delivery properties to the target cell may be decreased contrarily as compared to the case of not adding the unit α. Accordingly, there was a general technical disinclination in the field against increasing the addition ratio of the unit α, whereas the present inventors have found the following difficult-to-predict, micelle function-improving relationship: when the addition ratio of the unit α is further increased (contrary to the general technical inclination in the field), the delivery property of the polymer micelle pharmaceutical composition to the target cell is more certainly enhanced.

In the polymer micelle pharmaceutical composition 100, the backbone polymer unit α may be radially arranged in any appropriate state. For example, the backbone polymer unit α may be arranged in the state in which the hydrophilic polymer chain segment 11 is directed radially outward and the hydrophobic polymer chain segment 12 is directed radially inward.

The backbone polymer unit α preferably has a lower hydrophobicity than that of the backbone polymer unit β. The lower hydrophobicity may result from the hydrophilic group(s) of the side chains of the hydrophobic polymer chain segment.

When the hydrophobic polymer chain segment of the backbone polymer unit β is constituted of repeating units having side chains, the side chains of the hydrophobic polymer chain segment in the backbone polymer unit α may have a larger number of hydrophilic groups than the side chains of the hydrophobic polymer chain segment in the backbone polymer unit β.

Although the backbone polymer unit α is typically free of a target binding site and a drug, embodiments in which the backbone polymer unit α contains a target binding site and/or a drug are not excluded.

The backbone polymer unit α may be a block copolymer represented by the general formula: A₁−B₁. A₁ represents a polyethylene glycol chain segment, and B₁ represents a polyamino acid chain segment having at least one side chain which has a hydrophilic group; typically it contains one or more amino acid residues having a hydrophobic group in a side chain, and one or more amino acid residues having a hydrophilic group in a side chain. It should be noted that polyethylene glycol is hereinafter sometimes abbreviated as “PEG”.

Examples of the polyamino acid chain segment of the backbone polymer unit α having a hydrophobic group and a hydrophilic group may include: polyglutamic acid, or an ester or an amide derivative thereof; and polyaspartic acid, or an ester or an amide derivative thereof. Such ester or amide derivative may be formed through a reaction between a corresponding hydroxy compound or amino compound having the hydrophobic group and the hydrophilic group, and a reactive derivative (such as an ester) of polyglutamic acid or polyaspartic acid.

Examples of the hydrophobic group include hydrophobic organic groups. Examples of the hydrophobic organic groups include a C₄ to C₁₆ alkyl group having a linear, branched, or cyclic structure, a C₆ to C₂₀ aryl group, and a C₇ to C₂₀ aralkyl group or sterol residue. Preferred examples of the C₆ to C₂₀ aryl group and the C₇ to C₂₀ aralkyl group include a phenyl group, a naphthyl group, a tolyl group, a xylyl group, a benzyl group, and a phenethyl group, and of those, a more preferred example thereof is a benzyl group. In addition, as a sterol from which the sterol residue results, cholesterol, cholestanol, and dihydroxycholesterol are preferred, and cholesterol is more preferred.

Preferred specific examples of the backbone polymer unit α may be represented by the following general formulae (I) and (II). The polymer micelle pharmaceutical composition of the present teachings may include two or more kinds of the backbone polymer unit α.

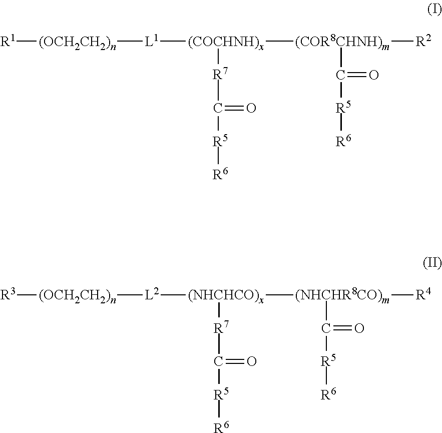

In each of the above-mentioned formulae:

R¹ and R³ each independently represent a hydrogen atom or a lower alkyl group, which is unsubstituted or is substituted with an optionally protected functional group;

R² represents a hydrogen atom, a saturated or unsaturated C₁ to C₂₉ aliphatic carbonyl group, or an arylcarbonyl group;

R⁴ represents a hydroxy group, a saturated or unsaturated C₁ to C₃₀ aliphatic oxy group, or an aryl-lower alkyloxy group;

each R⁵ represents —O— or —NH—;

10% to 90% of the total number (m+x) of the R⁶ groups is/are (a) hydrogen atom(s), and the remaining R⁶ group(s) is/are (a) hydrophobic organic group(s);

R⁷ and R⁸ each independently represent a methylene group or an ethylene group;

n is an integer of from 10 to 2,500;

x is an integer of from 10 to 300;

m is an integer of from 0 to 300 (provided that, when m is 1 or more, the binding order of the repeating units in which the number of repetitions is x and the repeating units in which the number of repetitions is m is arbitrary, and the R⁶ groups are each independently selected in each repeating unit in one block copolymer);

L¹ represents a linking group selected from the group consisting of —NH—, —O—, —O—Z—NH—, —CO—, —CH₂—, —O—Z—S—Z—, and —OCO—Z—NH— (where each Z independently represents a C₂ to C₆ alkylene group); and

L² represents a linking group selected from —OCO—Z—CO— and —NHCO—Z—CO— (where each Z independently represents a C₁ to C₆ alkylene group).

The percentage of hydrogen atoms in the total number (m+x) of R⁶ groups is, for example, 20% or more, and for example, 35% or more. The percentage is, for example, 80% or less, for example, 60% or less, and for example, 50% or less.

The above-mentioned n is preferably from 10 to 1,000, more preferably from 20 to 600, particularly preferably from 50 to 500. The above-mentioned x and m are each preferably from 20 to 200, more preferably from 30 to 100.

The binding order of the respective repeating units within the polyamino acid segment of the backbone polymer unit α represented by formulae (I) and (II) is arbitrary. The polyamino acid segment may have any of: a structure in which the binding order of the respective repeating units is random (random structure); a structure including a segment formed of repeating units with a number of repetitions of x and a segment formed of repeating units with a number of repetitions of m (block structure); and a structure in which m=0 (homopolymer structure).

Examples of the optionally protected functional group include a hydroxyl group, an acetal, a ketal, an aldehyde, a sugar residue, a maleimide group, a carboxyl group, an amino group, a thiol group, and an active ester. The hydrophilic polymer chain segment, in case R¹ and R³ each represent a lower alkyl group substituted with an optionally protected functional group, may be determined, for example, in accordance with the methods described in WO 96/33233 A1, WO 96/32434 A1, and WO 97/06202 A1. The lower alkyl group means a linear or branched alkyl group having, for example, 7 or less, preferably 4 or less carbon atoms, such as a methyl group, an ethyl group, a propyl group, an isopropyl group, a butyl group, or an isobutyl group.

The backbone polymer unit α may be obtained, for example, by coupling a polymer having a hydrophilic polymer chain and a polymer having a polyamino acid chain according to a known method, each of which has not been subjected to any treatment or has been purified so as to achieve a narrow molecular weight distribution as necessary. The block copolymer of the general formula (I) may also be formed, for example, by carrying out anionic living polymerization using an initiator capable of providing R¹ to form a polyethylene glycol chain, then introducing an amino group at the side of the growing end, and polymerizing an N-carboxylic anhydride (NCA) of a protected amino acid such as β-benzyl-L-aspartate or γ-benzyl-L-glutamate from the amino end.

A specific example of a method of manufacturing the backbone polymer unit α is described below. N-carboxy-β-benzyl-L-aspartate anhydride (BLA-NCA) or N-carboxy-γ-benzyl-L-glutamate anhydride (BLG-NCA) is added and subjected to reaction using, as an initiator, polyethylene glycol, which is protected at one end and has an amino group at the other end, such as MeO-PEG-CH₂CH₂CH₂—NH₂, in a dry organic solvent so as to achieve a desired degree of polymerization (number of amino acid units), whereby polyethylene glycol-co-polyaspartic acid benzyl ester or polyethylene glycol-co-polyglutamic acid benzyl ester may be prepared. In addition, the resultant block copolymer is acetylated at the end with acetyl chloride or acetic anhydride, then subjected to alkali hydrolysis to remove a benzyl group, and converted into polyethylene glycol-co-polyaspartic acid or polyethylene glycol-co-polyglutamic acid. After that, benzyl alcohol is added in an organic solvent so as to achieve a desired esterification ratio, and the reaction is carried out in the presence of a condensation agent such as N-N′-dicyclohexyl carbodiimide (DCC) or N-N′-diisopropyl carbodiimide (DIPCI), whereby a block copolymer partially having a benzyl ester may be prepared.

Further, when the reaction is performed using, for example, cholesterol in place of benzyl alcohol, polyethylene glycol-co-polyaspartic acid cholesterol ester and polyethylene glycol-co-polyglutamic acid cholesterol ester may be prepared.

Another specific example of a method of manufacturing the backbone polymer unit α is a method involving introducing a hydrophobic side chain through an amide bond. In the manufacturing method, polyethylene glycol-co-polyaspartic acid benzyl ester or polyethylene glycol-co-polyglutamic acid benzyl ester is acetylated at the end in the same manner as described above. Then, a benzyl group is removed by alkali hydrolysis and the generated carboxyl group is subjected to reaction with a hydrophobic side chain having an amino group. Alternatively, polyethylene glycol-co-polyaspartic acid benzyl ester or polyethylene glycol-co-polyglutamic acid benzyl ester and a compound having a primary amine are subjected to reaction and then subjected to aminolysis to convert an ester bond to an amide bond. This allows the introduction of a hydrophobic side chain through an amide bond. In addition, a poly(amino acid derivative) segment including a mixture of a hydrophobic side chain having a hydrophobic group whose end has been substituted by an amino group and a hydrophobic side chain without amino group substitution may also be obtained by adding a primary amine such as 1-octylamine to polyethylene glycol-co-polyaspartic acid benzyl ester in an organic solvent so as to achieve a desired amidation ratio, subjecting the mixture to reaction for a predetermined period of time, and then adding a large excess amount of 1,8-diaminooctane or the like to an unconverted benzyl ester.

Other preferred specific examples of the backbone polymer unit α may be represented by the following general formulae (III) and (IV).

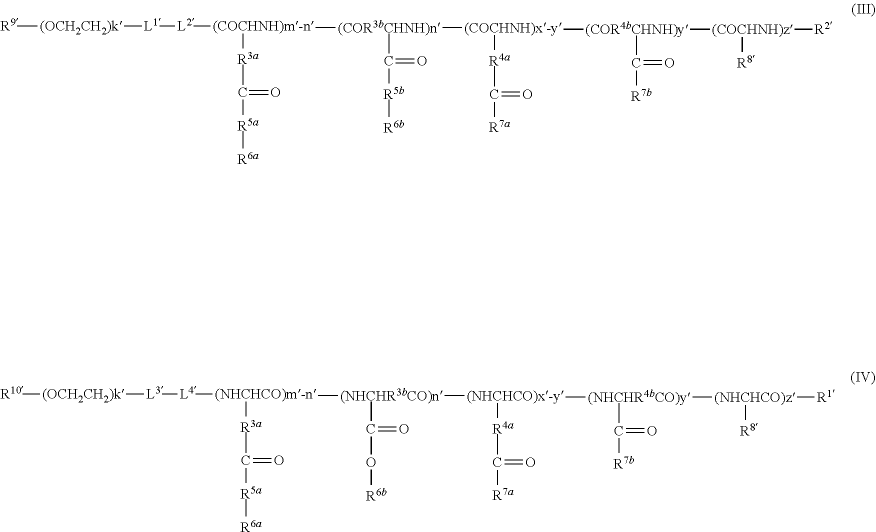

In the formulae:

R¹′ represents a hydroxyl group, an unsubstituted or substituted linear or branched alkyloxy group having 1 to 12 carbon atoms, an unsubstituted or substituted linear or branched alkenyloxy group having 2 to 12 carbon atoms, an unsubstituted or substituted linear or branched alkynyloxy group having 2 to 12 carbon atoms, or an unsubstituted or substituted linear or branched alkyl-substituted imino group having 1 to 12 carbon atoms;

R²′ represents a hydrogen atom, an unsubstituted or substituted linear or branched alkyl group having 1 to 12 carbon atoms, or an unsubstituted or substituted linear or branched alkylcarbonyl group having 1 to 24 carbon atoms;

each R^{3a}, R^{3b}, R^{4a}, and Rob independently represents a methylene group or an ethylene group;

each R^{5a} and R^{5b} independently represents —O— or —NH—;

each R^{6a} and R^{6b} independently represents a saturated or unsaturated linear or branched aliphatic hydrocarbon group having 6 to 27 carbon atoms, an aromatic hydrocarbon group having 6 to 27 carbon atoms, or a steryl group;

each R^{7a} and R^{7b} is independently selected from groups identical to or different from each other in the group consisting of the following groups:

—NH—(CH₂)ₚ₁—[NH—(CH₂)_{q1}—]ᵣ₁NH₂ (i);

—NH—(CH₂)ₚ₂—N[—(CH₂)_{q2}—NH₂]₂ (ii);

—NH—(CH₂)ₚ₃—N{[—(CH₂)_{q3}—NH₂][—(CH₂)_{q4}—NH—]ᵣ₂H} (iii); and

—NH—(CH₂)ₚ₄—N{—(CH₂)_{q5}—N[—(CH₂)_{q6}—NH₂]₂}₂ (iv)

where p1 to p4, q1 to q6, and r1 to r2 are each independently an integer of from 1 to 5;

R⁸′ represents a side chain of an amino acid selected from the group consisting of lysine, ornithine, arginine, homoarginine, and histidine;

m′ is an integer of from 5 to 80;

n′ is an integer of from 0 to m′;

x′ is an integer of from 0 to 20;

y′ is an integer of from 0 to x′;

z′ is an integer of from 0 to 20, provided that the total of x′ and z′ is 1 or more and 20 or less, the binding order of the respective repeating units is arbitrary, and each R^{6a}, R^{6b}, R^{7a}, R^{7b}, and R⁸′ may be arbitrarily selected in each amino acid residue in one polyamino acid;

L¹′ and L³′ are each independently —S—S— or a valence bond;

L²′ is —NH—, —O—, —O(CH₂)ₚ₅—NH—, or -L^{2a}-(CH₂)_{q7}-L^{2b}- where p5 and q7 are each independently an integer of from 1 to 5, L^{2a} is OCO, OCONH, NHCO, NHCOO, NHCONH, CONH, or COO, and L^{2b} is NH or O;

L⁴′ is —OCO—(CH₂)ₚ₆—CO—, —NHCO—(CH₂)ₚ₇—CO—, or -L^{4a}-(CH₂)_{q8}—CO— where p6, p7, and q8 are each independently an integer of from 1 to 5, and L^{4a} is OCONH, —CH₂NHCO—, NHCOO, NHCONH, CONH, or COO;

R⁹′ and R¹⁰′ are each independently a hydrogen atom, or an unsubstituted or substituted linear or branched alkyl group having 1 to 12 carbon atoms; and

k′ represents an integer of from 30 to 20,000.

In the above-mentioned formulae (III) and (IV), an alkyl moiety in the linear or branched alkyloxy group, alkyl-substituted imino group, and alkyl group each having 1 to 12 carbon atoms, which are defined by the R¹′ and R²′ groups, may be, for example, a methyl group, an ethyl group, a n-propyl group, an isopropyl group, a n-butyl group, a sec-butyl group, a tert-butyl group, a n-hexyl group, a decyl group, or an undecyl group. An alkenyl or alkynyl moiety in the linear or branched alkenyloxy group having 2 to 12 carbon atoms, or the linear or branched alkynyloxy group having 2 to 12 carbon atoms may be exemplified by an alkenyl or alkynyl moiety including a double bond or a triple bond in the alkyl group having 2 or more carbon atoms as exemplified above.

For such group or moiety, the substituent(s) in a “substituted” case may be exemplified by, but not limited to, a C₁₋₆ alkoxy group, an aryloxy group, an aryl C₁₋₃ oxy group, a cyano group, a carboxyl group, an amino group, a C₁₋₆ alkoxycarbonyl group, a C₂₋₇ acylamide group, a tri-C₁₋₆ alkyl siloxy group, a siloxy group, or a silylamino group, or may be exemplified by an acetalized formyl group, a formyl group, or a halogen atom (such as chlorine or fluorine). In this context, for example, the expression “C₁₋₆” means 1 to 6 carbon atoms and is used with the same meaning in the following description. In addition, an unsubstituted or substituted linear or branched alkyl moiety having 1 to 12 carbon atoms in the unsubstituted or substituted linear or branched alkylcarbonyl group having 1 to 24 carbon atoms may be selected with reference to the examples, and an alkyl moiety having 13 or more carbon atoms may be, for example, a tridecyl group, a tetradecyl group, a pentadecyl group, a nonadecyl group, a docosanyl group, or a tetracosyl group.

The binding order of the repeating units having the R^{3a}, R^{3b}, R^{4a}, and R^{4b} groups is arbitrary, and it may be a random structure, or it may be a block structure. When both of the R^{3a} and R^{3b} groups represent an ethylene group, typically polyamino acids are represented in which n′ represents an integer of 0 or a polyamino acid in which m′−n′ represents an integer of 0. The former represents, for example, poly-α-glutamic acid, which is obtained by the polymerization of an N-carboxylic anhydride of glutamic acid γ-benzyl ester, and the latter represents, for example, poly-γ-glutamic acid that strains of the genus Bacillus bacteria, such as Bacillus natto, produce. On the other hand, when both the R^{3a} and R^{3b} groups represent a methylene group, it is understood that the respective repeating units having those groups may coexist with each other. The same holds true for the R^{4a} and R^{4b} groups. It is preferred that the R^{3a} and R^{3b} groups each represent an ethylene group, and the R^{4a} and R^{4b} groups each represent a methylene group from the viewpoint of production efficiency.

In case the aliphatic hydrocarbon group is saturated in the definition of the R^{6a} and R^{6b} groups, the saturated aliphatic hydrocarbon group is equivalent to an alkyl group having 6 to 27 carbon atoms. Examples of the alkyl group include a hexyl group (such as a n-hexyl group), a decyl group, an undecyl group, a dodecyl group, a tridecyl group, a tetradecyl group, a pentadecyl group, a hexadecyl group, a heptadecyl group, an octadecyl group, a nonadecyl group, an icosyl group, an eicosyl group, a henicosyl group, a heneicosyl group, a docosyl group, a tricosyl group, a tetracosyl group, a pentacosyl group, a hexacosyl group, and a heptacosyl group. In case the aliphatic hydrocarbon group is unsaturated in the definition of the R^{6a} and R^{6b} groups, examples of the unsaturated aliphatic hydrocarbon group include groups each obtained by changing 1 to 5 carbon-carbon single bonds in the chain of the alkyl group exemplified above to carbon-carbon double bonds.

Examples of the aromatic hydrocarbon group having 6 to 27 carbon atoms in the definition of the R^{6a} and R^{6b} groups include an aryl group and an aralkyl group. Preferred specific examples of those groups include a phenyl group, a naphthyl group, a tolyl group, a xylyl group, a benzyl group, and a phenethyl group.

The sterol from which the steryl group in the definition of the R^{6a} and R^{6b} groups results means a natural, semisynthetic, or synthetic compound based on a cyclopentanone hydrophenanthrene ring (C₁₇H₂₈), and means derivatives thereof as well. Examples of the natural sterol may include, but are not limited to, cholesterol, cholestanol, dihydrocholesterol, cholic acid, campesterol, and sitosterol. Examples of the semisynthetic or synthetic sterol may include synthetic precursors of the natural sterols (encompassing compounds in which as necessary, if present, part or all of predetermined functional groups and hydroxy groups are protected with hydroxy-protecting groups known in the art, or carboxyl groups are protected by carboxyl protection). In addition, a C₁₋₁₂ alkyl group and/or a halogen atom (such as chlorine, bromine, or fluorine) may be introduced into the cyclopentanone hydrophenanthrene ring of the sterol derivative as long as the object of the present invention is not adversely affected. The ring system may be saturated, or may be partially unsaturated. The sterol from which the steryl group results is preferably a sterol originating from animal or vegetable oil such as cholesterol, cholestanol, dihydrocholesterol, cholic acid, campesterol, or sitosterol, more preferably cholesterol, cholestanol, or dihydroxycholesterol, particularly preferably cholesterol.

In the R^{7a} and R^{7b} groups, the groups selected from the following groups are defined:

—NH—(CH₂)ₚ₁—[NH—(CH₂)_{q1}—]ᵣ₁NH₂ (i);

—NH—(CH₂)ₚ₂—N[—(CH₂)_{q2}—NH₂]₂ (ii);

—NH—(CH₂)ₚ₃—N{[—(CH₂)_{q3}—NH₂][—(CH₂)_{q4}—NH—]ᵣ₂H} (iii); and

—NH—(CH₂)ₚ₄—N{—(CH₂)_{q5}—N[—(CH₂)_{q6}—NH₂]₂}₂ (iv);

preferably they are identical groups, and more preferably they are the group represented by formula (i). In addition, p1 to p4 and q1 to q6 are each independently preferably 2 or 3, more preferably 2. On the other hand, it is preferred that r1 and r2 are each independently an integer of from 1 to 3.

m′−n′ and n′ each represent the number of repetitions of hydrophobic amino acid residues, and x′−y′, y′, and z′ each represent the number of repetitions of cationic amino acid residues. The percentage of the number of repetitions (x′+z′) of cationic amino acid residues with respect to the total number of repetitions (m′+x′+z′) of amino acid residues is, for example, 20% or more, preferably 35% or more. The percentage is, for example, 80% or less, preferably 60% or less, more preferably 50% or less. Furthermore, x′ is preferably from 1 to 20, more preferably from 1 to 15, still more preferably from 1 to 10, and particularly preferably from 1 to 5. When x′ is 1 or more, the polyamino acid of the present invention includes at least the R^{7a} group or the R^{7b} group. When taken up into an endosome (pH 5.5) and exposed to a lower pH, the backbone polymer unit α having such structure undergoes further protonation of the cationic polyamino acid and exerts a buffer effect (or proton sponge effect), and thus the endosomal escape of the drug to be delivered can be facilitated by the polymer micelle pharmaceutical composition being simultaneously taken up by endocytosis.

The binding order of the respective repeating units within the polyamino acid segment of the backbone polymer unit α represented by formulae (III) and (IV) is arbitrary, and it may be a random structure, or it may be a block structure. When the polyamino acid segment is a block type containing the segment formed of the cationic amino acid residues and the segment formed of the hydrophobic amino acid residues, large functional unit groups in which functional units capable of inducing endocytosis and functional units capable of inducing endosomal escape are grouped, respectively, are formed, and hence the functions can be more certainly induced.

The above-mentioned L¹′ and L³′ are each independently —S—S— or a valence bond. On the other hand, L²′ is —NH—, —O—, —O(CH₂)ₚ₅—NH—, or -L^{2a}-(CH₂)_{q7}-L^{2b}- where p5 and q7 are each independently an integer of from 1 to 5, L^{2a} is OCO, OCONH, NHCO, NHCOO, NHCONH, CONH, or COO, and L^{2b} is NH or O. In addition, L⁴′ is —OCO— (CH₂)ₚ₆—CO—, —NHCO—(CH₂)ₚ₇—CO—, or -L^{4a}-(CH₂)_{q8}—CO— where p6, p7, and q8 are each independently an integer of from 1 to 5, and L^{4a} is OCONH, —CH₂NHCO—, NHCOO, NHCONH, CONH, or COO. In the definition, the combination of L¹′ and L²′, and the combination of L³′ and L⁴′ each need to be such that the groups may together form one linking group. When, for example, L²′ is —NH—, L¹′ is not —S—S— but rather is a valence bond. A combination that forms a linking group in case L¹′ or L³′ is —S—S— is preferred as the combination.

Examples of the linear or branched alkyl group having 1 to 12 carbon atoms in the definition of the R⁹′ and R¹⁰′ groups include the same groups as the alkyl moieties of the linear or branched alkyloxy group, alkyl-substituted imino group, and alkyl group each having 1 to 12 carbon atoms in the definition of the R¹′ and R²′ groups. In addition, the same applies to its substituent.

k′, which represents the number of repetitions of ethylene glycol (or oxyethylene), represents an integer of, for example, from 30 to 20,000, preferably from 40 to 2,000, still more preferably from 50 to 1,000.

The backbone polymer unit α may be formed by, for example, coupling the cationic polyamino acid and a hydrophilic polymer by a known method as they are, or as necessary, after purification to narrow their molecular weight distributions. In addition, for example, the block copolymer of the general formula (III) may be produced by: performing anionic living polymerization through the use of an initiator capable of providing R⁹′ to form a polyethylene glycol chain; then introducing an amino group to the growing end side; polymerizing, from the resultant amino end, an N-carboxylic anhydride (NCA) of a protected amino acid, such as β-benzyl-L-aspartate, γ-benzyl-L-glutamate, or Nε-Z-L-lysine; and introducing a cationic group into a side chain of the resultant polyamino acid. It should be noted that a structural change due to nucleophilic attack by polyamine (such as the formation of an imide ring through the dealcoholization of an amino acid ester residue) may occur in some amino acid ester residues during the process of the synthesis of the cationic polyamino acid, and a block copolymer containing a residue which has undergone such structural change is herein also regarded as being included in the general formulae (III) and (IV). In addition, some NH groups and NH₂ groups in the cationic amino acid residues may be converted into salts (mainly hydrochlorides) owing to the use of an acid (mainly hydrochloric acid) in the synthesis process, and a block copolymer containing such structure is herein also regarded as being included in the general formulae (III) and (IV).

The polyamino acid chain segment of the backbone polymer unit α represented by the general formulae (III) and (IV) may be produced by, for example: polymerizing an N-carboxylic anhydride (NCA) of a protected amino acid known per se, such as β-benzyl-L-aspartate, γ-benzyl-L-glutamate, or Nε-Z-L-lysine to produce a polyamino acid ester; and then performing aminolysis using a polyamine corresponding to the R^{7a}, R^{7b}, and R⁸′ groups to introduce cationic groups into side chains of polyamino acid.

In one embodiment, γ-benzyl-L-glutamate is polymerized and then β-benzyl-L-aspartate is polymerized, followed by a reaction with an amine compound, such as diethylenetriamine (DET). Thus, poly(β-benzyl-L-aspartate) is preferentially subjected to an ester-amide exchange reaction, with the result that an amine residue, such as a DET group, is introduced into an aspartic acid side chain. As a result, a block-type cationic polyamino acid may be formed of an aspartic acid-derived cationic amino acid residue segment having a cationic group introduced into a side chain and a glutamic acid-derived hydrophobic amino acid residue segment having a benzyl group introduced into a side chain. On the other hand, when β-benzyl-L-aspartate and γ-benzyl-L-glutamate are simultaneously polymerized, followed by a reaction with an amine compound, such as diethylenetriamine (DET), a random-type cationic polyamino acid may be formed in which an aspartic acid-derived cationic amino acid residue having a cationic group introduced into a side chain and a glutamic acid-derived hydrophobic amino acid residue having a benzyl group introduced into a side chain are arbitrarily arranged.

It should be noted that a structural change due to nucleophilic attack by an amine (such as the formation of an imide ring through the dealcoholization of an amino acid ester residue) may occur in some amino acid ester residues during the synthesis process, and block copolymers containing one or more residues which has (have) undergone such structural change are herein also regarded as being included in the general formulae (III) and (IV). In this case, the number of residues which have undergone the structural change is not included in either of the number of cationic polyamino acid residues or the number of hydrophobic amino acid residues. In addition, some NH groups and NH₂ groups in the cationic amino acid residues may be converted into salts (mainly hydrochlorides) owing to the use of an acid (mainly hydrochloric acid) in the synthesis process, and block copolymers containing one or more residues which has (have) undergone such structural change are herein also regarded as being included in the general formulae (III) and (IV). That is, some NH groups and NH₂ groups in the R^{7a}, R^{7b}, and R⁸′ groups may be converted into salts (such as hydrochlorides).

3. Backbone Polymer Unit β

As was mentioned above, the backbone polymer unit β has the hydrophilic polymer chain segment and the hydrophobic polymer chain segment, and is arranged in the state in which the hydrophilic polymer chain segment is directed radially outward and the hydrophobic polymer chain segment is directed radially inward. The polymer micelle pharmaceutical composition may contain two or more kinds of the backbone polymer unit β.

The backbone polymer unit β may have a drug bound to the hydrophobic polymer chain segment.

The backbone polymer unit β preferably has a higher hydrophobicity than that of the backbone polymer unit α. The higher hydrophobicity may result from the drug. For example, the backbone polymer unit β, even while having more hydrophilic groups than the backbone polymer unit α, may have a higher hydrophobicity than the backbone polymer unit α owing to the hydrophobic properties of the drug.

The backbone polymer unit β may be a complex of a drug and a block copolymer, the complex being represented by the general formula: A₂-B₂(-D). A₂ represents a polyethylene glycol chain segment, B₂ represents a polyamino acid chain segment, and D represents the drug.

Examples of the polyethylene glycol chain segment and the polyamino acid chain segment of the backbone polymer unit β may include similar ones to those in the case of the backbone polymer unit α. It should be noted that the polyamino acid chain segment of the backbone polymer unit β does not need to have any hydrophilic group in a side chain.

The backbone polymer unit β may optionally contain a target binding site, but typically contains a drug while being free of a target binding site. When the polymer micelle pharmaceutical composition is prepared such that the drug and the target binding site are contained in different polymer units, in case the micelle structure has collapsed before the polymer micelle pharmaceutical composition migrates within the bloodstream to the vicinity of the target cell, the drug (still bound to the polymer unit) can be discharged out of the body through metabolism, and hence the occurrence of side effects is easily avoided. Further, it is not necessary to bind both the compound having a target binding site and the drug to the same block copolymer, and hence the deactivation of the drug or the compound for target binding during synthesis is easily avoided. It should be noted that when the target binding site is loaded into the backbone polymer unit β, the target binding site is preferably bound to a projecting end of the hydrophilic polymer chain segment.

Preferred specific examples of the backbone polymer unit β include the block copolymers represented by the general formulae (I) and (II). It should be noted that with regard to the R⁶ groups in the general formulae (I) and (II), 10% or more of the total number (m+x) of R⁶ groups is/are each a residue of a drug, which may have a linking group, and the remaining group(s), if present, is/are a hydrogen atom or a hydrophobic organic group. The percentage of the residues of the drug, which may have a linking group, with respect to the total number (m+x) is preferably from 10% to 100%, more preferably from 10% to 70%. The R⁶ groups are each independently selected in each amino acid unit in one block copolymer. The binding order of the respective repeating units within the polyamino acid segment of the backbone polymer unit β is arbitrary.

Examples of the drug may include nucleic acids (such as a nucleoside, a DNA, a RNA, a siRNA, and a microRNA), a nucleic acid derivative, a vaccine, an antibody having pharmacological activity (the so-called antibody medicine), docetaxel, a camptothecin, epothilone A, epothilone B, epothilone C, epothilone D, and derivatives of these epothilones, temsirolimus, everolimus, trabectedin, vorinostat, octreotide acetate, mitoxantrone, vincristine, cephalexin, cefaclor, ampicillin, bacampicillin, amoxicillin, kanamycin, amikacin, arbekacin, dibekacin, sisomicin, tobramycin, erythromycin, clarithromycin, rokitamycin, chloramphenicol, vancomycin, fluconazole, vidarabine, acyclovir, didanosine, zidovudine, zalcitabine, lamivudine, zanamivir, oseltamivir, lopinavir, and ritonavir. Examples of the derivatives of the epothilones may include patupilone, ixabepilone, BMS-310705, KOS-862, and ZK-EPO.

Examples of the nucleic acid derivative may include gemcitabine, nelarabine, clofarabine, decitabine, streptozocin, doxifluridine, and fludarabine. The nucleic acid may be bound to the backbone polymer unit β, for example, through a covalent bond and/or an electrostatic bond. The nucleic acid derivative may be a salt, but when the nucleic acid derivative is bound to the backbone polymer unit β through an ester bond, it is preferred that the nucleic acid derivative is not a salt.

The drug and the block copolymer unit may be bound, for example, through a covalent bond and/or an electrostatic bond. Examples of the covalent bond include a single (covalent) bond and a divalent linking group. An example of the divalent linking group is a divalent linking group which has 0 to 5 carbon atoms and may contain an amide bond, an ester bond, an ether bond, and/or a hydrazide bond. The divalent linking group is preferably an ester bond, an amide bond, or a hydrazide bond.

With regard to the binding, one of the drugs and one block copolymer unit may be bound through one covalent bond or electrostatic bond, or may be bound through two or more covalent bonds and/or electrostatic bonds. Further, one of the drugs and two or more block copolymer units may be bound through two or more covalent bonds and/or electrostatic bonds (that is, two or more block copolymer units are bound in a state of being cross-linked through one drug).

When the drug has a plurality of hydroxy groups, the backbone polymer unit β may have a structure in which one or more of the hydroxy groups are ester-bonded to carboxyl groups of a polyamino acid side chain. Herein, a structure in which one drug is ester-bonded to a plurality of carboxyl groups in the polyamino acid side chain, and a structure in which two or more block copolymer moieties are cross-linked through one drug are also regarded as being included in the backbone polymer unit β.

Examples of the ester bond include: an ester bond formed through a reaction between a drug having a hydroxy group, and a block copolymer unit having a carboxyl group; and an ester bond formed through a reaction between a drug having a carboxyl group, and a block copolymer unit having a hydroxy group.

Examples of the amide bond include: an amide bond formed through a reaction between a drug as an amine, and a block copolymer unit having an ester group; and an amide bond formed through a reaction between a drug having an amino group, and a block copolymer unit having a carboxyl group.

An example of the hydrazide bond is a hydrazide bond formed by the binding of a drug having a ketone structure to a hydrazide group of a block copolymer unit.

4. Backbone Polymer Unit γ

The polymer micelle pharmaceutical composition may further include a block copolymer unit γ (backbone polymer unit γ) having a hydrophilic polymer chain segment having a target binding site bound thereto, and a hydrophobic polymer chain segment, and being arranged together with the block copolymer units α and β in the state in which the hydrophilic polymer chain segment is directed radially outward and the hydrophobic polymer chain segment is directed radially inward. The backbone polymer unit γ may be arranged in the state in which the target binding site is directed outward.

The backbone polymer unit γ desirably has such a degree of hydrophobicity (steadiness) that the particle backbone of the polymer micelle can be formed; more specifically, it desirably has a degree of hydrophobicity (hydrophobic structure) of almost the same amount as the backbone polymer unit β.

Examples of the hydrophilic polymer chain segment and the hydrophobic polymer chain segment of the backbone polymer unit γ may include similar ones to the respective segments in the case of the backbone polymer unit α. It should be noted that the hydrophobic polymer chain segment of the backbone polymer unit γ does not need to have any hydrophilic group in a side chain.

The backbone polymer unit γ may be a complex of a compound having a target binding site and a block copolymer, the complex being represented by the general formula: Z-A₃-B₃. Z represents the compound having a target binding site, A₃ represents a polyethylene glycol chain segment, and B₃ represents a polyamino acid chain segment.

An example of the compound having a target binding site may be, as described above, a protein, a peptide, or a sugar chain which is capable of forming a binding pair with a substance of biological and viral origin. Examples of such protein may include: an antibody and a fragment thereof which bind to the substance of biological and viral origin; transferrin; and epidermal growth factor (EGF). Examples of the antibody may include antibodies capable of recognizing antigens including receptors and cell surface antigens, such as EGFR, Her2, CD20, VEGFR, and CD52, highly expressed on surfaces of medication targets typified by cancer cells. The antibody may be a monoclonal antibody, or may be a polyclonal antibody. The fragment of the antibody only needs to have a length which allows specific recognition of an antigen, and examples thereof may include (Fab′)2 and Fab. Examples of the peptide may include insulin, LHRH, IGF, and derivatives thereof. Examples of the sugar may include sugars having glucose, mannose, galactose, and fucose residues. The compound having a target binding site may be a compound capable of exhibiting pharmacological activity in itself (such as an antibody drug or a vaccine).

When a target with which the compound having a target binding site is to form a binding pair is a substance of viral origin, cells to which the substance is to be supplied are in a state in which cell death has occurred through the disruption of the cell membrane by a virus infecting the cells, and hence the polymer micelle pharmaceutical composition cannot be taken up into the cells through endocytosis. Therefore, herein, when the target is the substance of viral origin, cells present in proximity of the target are regarded as cells to which the target is to be supplied. When the substance of viral origin is present extracellularly, it is highly probable that such cells in the surroundings are infected with the virus, and hence the supply of the drug to the surrounding cells is of significance.

Examples of the polyethylene glycol chain segment and the polyamino acid chain segment of the backbone polymer unit γ may include similar ones to those in the cases of the backbone polymer units α and β, and the same applies to the formation method therefor.

Preferred specific examples of the backbone polymer unit γ include the block copolymers represented by the above-mentioned general formulae (I) and (II). The polymer micelle pharmaceutical composition of the present teachings may contain two or more kinds of the backbone polymer unit γ. It should be noted that in the general formulae (I) and (II), R¹ and R³ each represent a compound having a target binding site. Further, each R⁶ group represents a hydrogen atom or a hydrophobic organic group, and the percentage of the hydrogen atoms with respect to the total number (m+x) is, for example, from 0% to 60%, preferably from 0% to 20%, and the remaining group(s) is (are) a hydrophobic organic group.

The backbone polymer unit γ may be formed by a condensation or addition reaction of a block copolymer having, in the α-end of its polyethylene glycol chain segment, a linking group such as a hydroxyl group, a carboxyl group, an aldehyde group, an amino group, a mercapto group, or a maleimide group, and the compound having a target binding site.

5. Production Method for Polymer Micelle Pharmaceutical Composition

The polymer micelle pharmaceutical composition of the present teachings may be formed by, for example: dissolving the backbone polymer unit α and the backbone polymer unit β, or the backbone polymer unit α, the backbone polymer unit β, and the backbone polymer unit γ in an organic solvent; mixing the contents to achieve homogenization; subjecting the resultant solution to evaporation under reduced pressure; adding water to the resultant film of polymers; and mixing the contents to allow the polymers to self-assemble into a micellar form. In addition, for example, the polymer micelle pharmaceutical composition of the present teachings may be formed by mixing those backbone polymer units in an aqueous solution to allow the backbone polymer units to self-assemble into a micellar form. Further, for example, the polymer micelle pharmaceutical composition of the present teachings may also be formed by: dissolving the backbone polymer unit α, the backbone polymer unit β, and a precursor of the backbone polymer unit γ in an organic solvent; mixing the contents to achieve homogenization; subjecting the resultant solution to evaporation under reduced pressure; adding water to the resultant film of polymers; mixing the contents to allow the polymers to self-assemble into a micellar form; and then binding the compound having a target binding site to the α-end of the hydrophilic segment of the precursor of the backbone polymer unit γ to produce the backbone polymer unit γ. Further, for example, the polymer micelle pharmaceutical composition of the present teachings may also be formed by: mixing those backbone polymer units in an aqueous solution to allow the backbone polymer units to self-assemble into a micellar form; and then binding the compound having a target binding site to the α-end of the hydrophilic segment of the precursor of the backbone polymer unit γ to produce the backbone polymer unit γ. Examples of the organic solvent include methanol and acetone. The aqueous solution may be formed by, for example, adding a water-miscible organic solvent, such as ethanol or dimethyl sulfoxide, and a known buffer to purified water.

### EXAMPLES

Hereinafter, Examples of the present teachings are more specifically described. The present invention is by no means limited by these Examples.

### Example 1

A Herceptin-bound docetaxel (DTX) micelle, which is a polymer micelle formulation having HERCEPTIN® (Genentech, commonly known as trastuzumab) as the compound with a target binding site and encapsulating docetaxel (DTX) as the drug, was formed as described below.

Backbone Polymer Unit α

A polyethylene glycol-polyglutamic acid benzyl ester copolymer (one end of polyglutamic acid is acetylated; the average molecular weight (Da) of the PEG is 10,000; the average number of glutamic acid residues is 40; 60% of the hydrogen atoms of the carboxylic acids in the side chains are substituted with phenyl groups. The copolymer is hereinafter sometimes referred to as “PEG-PBLG (OBn: 60%)”.) was used as the backbone polymer unit α.

Backbone Polymer Unit β

PEG-pAsp-DTX as the backbone polymer unit β was produced as described below. 500 mg of a polyethylene glycol-polyaspartic acid block copolymer (PEG-pAsp-Ac) in a state of having one end of polyaspartic acid acetylated was dissolved in 10 mL of anhydrous DMF (Kanto Chemical Co., Inc.), and then 1.06 g of docetaxel (ScinoPharm Taiwan Ltd.) was further added. It should be noted that the PEG-pAsp-Ac has an average molecular weight (Da) of the PEG of 10,000, has an average number of the aspartic acid residues of 40, and has carboxylic acid as the side chain of the aspartic acids. Subsequently, 160 mg of 4-dimethylaminopyridine (Wako Pure Chemical Industries, Ltd.) and 210 μL of N,N′-diisopropylcarbodiimide (Kokusan Chemical Co., Ltd.) were added in the stated order, and the mixture was stirred at room temperature overnight. The thus-obtained reaction liquid was added dropwise to 500 mL of a mixed solution of hexane and ethyl acetate (volume ratio: 1:1) to crystallize a polymer, and then the polymer was collected by filtration under reduced pressure. The polymer collected by filtration was suspended in 100 mL of purified water to form a polymer micelle, followed by ultrafiltration (Labscale TFF System manufactured by Merck Millipore Corporation, molecular weight cutoff value: 100,000, 5-fold dilution and then concentration to 100 mL). This ultrafiltration operation was repeated five times, followed by lyophilization. The polymer obtained by the lyophilization was added dropwise, in a state of being dissolved in 10 mL of anhydrous DMF, to 500 mL of a mixed solution of hexane and ethyl acetate (volume ratio: 1:1) to crystallize the polymer, and then the polymer was collected by filtration under reduced pressure. The polymer collected by filtration was washed by being added, while in a powder state, to 100 mL of a mixed solution of hexane and ethyl acetate (volume ratio: 1:1), and was then collected by filtration under reduced pressure. The polymer collected by filtration was dried under reduced pressure at room temperature overnight to provide 530 mg of PEG-pAsp-DTX as a pale yellow powder.

1 mg of PEG-pAsp-DTX was dissolved in 10 mL of a mixed solution of purified water and ethanol (volume ratio: 1:1), and its docetaxel content was measured based on absorbance with respect to light having a wavelength of 233 nm and was found to be 14.3 molecules per polymer. The PEG-pAsp-DTX is in the state in which docetaxel is bound to PEG-pAsp-Ac through an ester bond. The chain length of the PEG is 10 kDa.

Maleimide-Bound Polymer

A maleimide-polyethylene glycol-polyglutamic acid copolymer having a maleimide group at a PEG end (one end of polyglutamic acid is acetylated; the average molecular weight (Da) of the PEG is 10,000; the average number of glutamic acid residues is 40; and all hydrogen atoms of the carboxylic acids in the side chains are substituted with phenyl groups.) was prepared. The copolymer is hereinafter sometimes referred to as “Maleimide-PEG-PBLG”.

Formation of Micelle

PEG-PBLG (OBn: 60%), PEG-pAsp-DTX, and Maleimide-PEG-PBLG were precisely weighed at a weight ratio of 4:5:1 in separate vials, and were each dissolved by the addition of methanol or acetone. The respective solutions were mixed together, and then the solvent was evaporated with a rotary evaporator (manufactured by Büchi, ROTAVAPOR® R-205, Vac V-513) to form a film of mixed polymers, which was further dried for one day and night. 100 mM PBS (pH 7.4) was added to disperse the film, and the resultant was subjected to high-pressure dispersion treatment using a NanoVater® (manufactured by Yoshida Kikai Co., Ltd., NM2-L200) to provide a micelle. The micelle fraction contains a micelle in which PEG-PBLG (OBn: 60%), PEG-pAsp-DTX, and Maleimide-PEG-PBLG are radially arranged.

Reaction Between HERCEPTIN® and Maleimide

Purified water was added to a vial containing HERCEPTIN at a molar ratio of 0.4 with respect to Maleimide-PEG-PBLG to prepare a Herceptin solution. The Herceptin solution was adjusted to have a final concentration of borate buffer of 50 mM and a final concentration of EDTA of 1 mM, and 10 mg/mL Traut's Reagent (Pierce Biotechnology Inc.) was further added. After that, the mixture was left to stand still at 30° C. for 45 minutes. Subsequently, the thus-obtained reaction liquid was purified by gel filtration [PD-10 manufactured by GE Healthcare Life Sciences, eluent: 100 mM sodium phosphate buffer (pH 7.4), 1 mM EDTA], and a high-molecular-weight fraction was collected.

The collected liquid and the micelle fraction were mixed, and the mixture was left to stand still at 30° C. for 2 hours to subject the maleimide group of Maleimide-PEG-PBLG and HERCEPTIN to reaction. Thus, a micelle containing a Herceptin-bound polymer as the backbone polymer unit γ was formed. The thus-obtained reaction liquid was purified by ultrafiltration (manufactured by Merck Millipore Corporation, AMICON® Ultra-15, membrane fraction: 300,000) to remove unreacted HERCEPTIN. Thus, a solution containing a Herceptin-bound docetaxel micelle having PEG-PBLG (OBn: 60%) was obtained. The docetaxel content in the micelle solution was 2.154 mg/mL.

### Comparative Example 1

A solution containing a Herceptin-bound docetaxel micelle was obtained in the same manner as in Example 1 except that PEG-pAsp-DTX and Maleimide-PEG-PBLG were used at a weight ratio of 9:1, and PEG-PBLG (OBn: 60%) was not used. The docetaxel content in the micelle solution was 1.273 mg/mL.

### Comparative Example 2

A 10% sucrose solution containing a docetaxel micelle was obtained by using only PEG-pAsp-DTX. The docetaxel content in the micelle solution was 3.486 mg/mL.

Cytotoxicity Test 1

The cytotoxicity of the Herceptin-bound docetaxel micelle having PEG-PBLG (OBn: 60%) of Example 1 was compared to the cytotoxicity of each of the Herceptin-bound docetaxel micelle of Comparative Example 1 and the docetaxel micelle of Comparative Example 2. The cytotoxicity of each of the samples was evaluated as described below based on the WST method using human prostate cancer-derived PC-3 cells (HER2-negative) purchased from ATCC through Summit Pharmaceuticals International Corporation.

The PC-3 cells were seeded in a 96-well plate in a state of being suspended in 90 μL of a medium so that about 5,000 of the cells were contained per well, and the cells were cultured under a 5% CO₂ atmosphere at 37° C. overnight. RPMI 1640 (Gibco®, Invitrogen) and 10% FBS (biowest) were used as the medium. Subsequently, a solution prepared by diluting any one of the samples with the medium so as to have any of various docetaxel concentrations was added to each well (10 μL per well), and culturing was performed under a 5% CO₂ atmosphere at 37° C. for 72 hours. After that, WST Reagent (Dojindo Laboratories) was added (10 μL per well), and culturing was continued under a 5% CO₂ atmosphere at 37° C. for about 2 hours. Absorbance with respect to light having a wavelength of 450 nm (Abs450) was measured for each well, and the cell growth rate (% Cell Growth) was calculated on the basis of the following equation. It should be noted that “Abs450 value of control” in the equation means the absorbance obtained from a well in which culturing as described above was performed using a culture solution containing no docetaxel.

$$\% cell growth = \frac{( \begin{matrix}Abs 450 value after \\ addition of sample liquid\end{matrix} ) - ( Abs 450 value of blank )}{( Abs 450 value of control ) - ( Abs 450 value of blank )} \times 100$$

The docetaxel concentration was set to 0.05 ng/mL, 0.14 ng/mL, 0.41 ng/mL, 1.23 ng/mL, 3.7 ng/mL, 11.1 ng/mL, 33.3 ng/mL, or 100 ng/mL. The results were subjected to logarithmic approximation by placing the cell growth rate (%) on the vertical axis and the docetaxel concentration (ng/mL) on the horizontal axis, to calculate an IC25 (25% inhibition concentration (ng/mL)). The results are shown in Table 1.

It should be noted that specific examples of data on Cytotoxicity Test 1 are described below.

(When the Concentration in Terms of Docetaxel was 3.7 ng/mL)

The cell growth rate of the PC-3 cells (%) was 81.9% (Example 1), 106% (Comparative Example 1), or 109% (Comparative Example 2).

(When the Concentration in Terms of Docetaxel was 11.1 ng/mL)

The cell growth rate of the PC-3 cells (%) was 46.5% (Example 1), 59.6% (Comparative Example 1), or 62.4% (Comparative Example 2).

(When the Concentration in Terms of Docetaxel was 33.3 ng/mL)

The cell growth rate of the PC-3 cells (%) was 24.6% (Example 1), 30.0% (Comparative Example 1), or 33.9% (Comparative Example 2).

Cytotoxicity Test 2

The IC₂₅ (25% inhibition concentration (ng/mL)) was calculated by performing the same experiment as Cytotoxicity Test 1 except that: human stomach cancer-derived NCI-N87 cells (HER2-positive) purchased from ATCC through Summit Pharmaceuticals International Corporation were used; the number of NCI-N87 cells was set to about 10,000 cells per well; the docetaxel concentration was set to 0.5 ng/mL, 1.4 ng/mL, 4.1 ng/mL, 12.4 ng/mL, 37.0 ng/mL, 111.1 ng/mL, 333.3 ng/mL, or 1,000.0 ng/mL; and the cells were cultured under a 5% CO₂ atmosphere at 37° C. for 1 hour, the medium was then removed from the wells, the wells were washed with phosphate buffered saline (PBS) twice, a fresh medium was then added (100 μL per well), and culturing was continued until the total culture time became 72 hours. The results are shown in Table 1.

It should be noted that specific examples of data on Cytotoxicity Test 2 are described below.

(When the Concentration in Terms of Docetaxel was 37.0 ng/mL)

The cell growth rate of the NCI-N87 cells (%) was 82.6% (Example 1) or 92.9% (Comparative Example 1).

(When the Concentration in Terms of Docetaxel was 111.1 ng/mL)

The cell growth rate of the NCI-N87 cells (%) was 73.0% (Example 1) or 88.0% (Comparative Example 1).

TABLE 1

| | | Comparative | Comparative |
|---|---|---|---|
| | Example 1 | Example 1 | Example 2 |
| PC-3 cells | 3.3 | 8.4 | 9.2 |
| NCI-N87 cells | 88.1 | 403.4 | N.D. |

The results revealed that the polymer micelle pharmaceutical composition containing PEG-PBLG (OBn: 60%) (Example 1) exhibited a cytocidal effect in a much lower concentration range as compared to the polymer micelle pharmaceutical compositions free of PEG-PBLG (OBn: 60%) (Comparative Examples 1 and 2). The results were observed irrespective of the presence or absence of an antigen for Herceptin in the cells. Accordingly, it is considered that the favorable effect based on the admixing of PEG-PBLG (OBn: 60%) is exhibited irrespective of the presence or absence of the backbone polymer unit γ having a target binding site. Further, because the maintenance/collapsing of the particle structure of the polymer micelle pharmaceutical composition is considered to be the essence of the mechanism of action, it is considered that there is no particular restriction on the drug to be encapsulated.

Anti-Tumor Effect Confirmation Test

HER2-positive human stomach cancer NCI-N87 cells were purchased from ATCC through Summit Pharmaceuticals International Corporation. The cells were cultured using an RPMI 1640+10% FBS medium under 5% CO₂ at 37° C., and grown until the number of cells reached a number required for transplantation. The cells were suspended in saline to prepare a suspension having a concentration of 6.0×10⁷ cells/mL. To the cell suspension, an equal amount of a substrate for cell culture (manufactured by Nippon Becton Dickinson Company, Ltd., trade name: BD Matrigel®) cooled with ice was added (cell concentration: 3.0×10⁷ cells/mL). The resultant was inoculated subcutaneously into the right abdomens of male nude mice (Balb nu/nu, 6-weeks old, provided by Charles River Laboratories Japan, Inc.) at 3.0×10⁶ cells/100 μL per mouse. After that, the nude mice were reared for 15 days, and a medicament was administered when the tumor volume reached about 130 mm³. The micelle of Example 1 and the micelle of Comparative Example 1 were each administered into the tail vein in a single dose of 7 mg/kg in terms of docetaxel. Further, as Comparative Example 3, a 10% sucrose solution was administered as the medium. In each of the Example and Comparative Examples, five animals were used as a group. Immediately before the administration of the micelle formulation and on day 10 after the administration, the tumor volume and the body weight of each of the nude mice were measured, and relative values of the tumor volume and the body weight on day 10 after the administration with respect to those immediately before the administration of the micelle formulation were measured. The results are shown in Table 2.

TABLE 2

| | | Comparative | Comparative |
|---|---|---|---|
| | Example 1 | Example 1 | Example 3 |
| Relative tumor volume | 119.6% | 147.5% | 174.8% |
| Relative body weight | 104.6% | 104.3% | 106.3% |

From the results shown in Table 2, it was found that, when the polymer micelle pharmaceutical composition containing PEG-PBLG (OBn: 60%) (Example 1) was administered, while a state was achieved in which the changes in mouse body weight did not differ, the growth of the human stomach cancer NCI-N87 cells could be remarkably suppressed as compared to the case of administering the polymer micelle pharmaceutical composition free of PEG-PBLG (OBn: 60%) (Comparative Example 1) and the case of administering the medium (Comparative Example 3).

### Test Example 1

Preparation of Alexa 488-labeled SH-modified Herceptin 1 mL of a Herceptin formulation (22 mg/mL) was dispensed into a 1.5-mL microtube. In order to remove histidine contained in the formulation, the formulation was purified by gel filtration (manufactured by GE Healthcare Japan, trade name: “PD-10”). The concentration of the antibody was measured using a spectrophotometer at 280 nm and adjusted to 10 mg/mL. An Alexa 488 labeling kit (manufactured by Life Technologies, trade name: “Alexa Fluor® 488 Protein Labeling Kit”) was used to label an amino group in the antibody in accordance with the protocol described in the attached document. Unreacted labeling reagent (fluorescein derivative) was removed using PD-10, and the concentration of the antibody was calculated again with the spectrophotometer (280 nm and 494 nm). 20 equivalents of a 10 mg/mL Traut's Reagent solution (1 mM EDTA borate buffer (pH 8.0)) was added with respect to 100 parts by weight of the Alexa 488-labeled Herceptin. An SH modification reaction with Traut's Reagent was performed in a water bath at 30° C. for 45 minutes, and after the completion of the reaction, PD-10 was used to remove the excess of Traut's Reagent. The Alexa 488-labeled SH-modified Herceptin in this state is hereinafter denoted as “Alexa-Herceptin-SH”.

Formation of Micelle

90 mg of PEG-PBLG (OBn: 100%) and 10 mg of Maleimide-PEG-PBLG (content: 9:1 in the stated order) were weighed, and each polymer was dissolved with methanol or acetone. The respective polymer solutions were mixed and homogenized in a 100-mL recovery flask, and the organic solvent was evaporated with an evaporator to provide an amorphous film of mixed polymers. 10 mL of PBS was added to about 100 mg of the film of the polymers obtained, and the contents were dissolved using an ultrasonic device. The solution was subjected to polymer micellization using a high-pressure disperser (manufactured by Yoshida Kikai Co., Ltd.: NanoVater® NM2-L200) to provide a 10 mg/mL mixed polymer micelle solution. The micelle solution obtained by the high-pressure dispersion treatment was measured for its particle diameter with a dynamic light scattering photometer.

Reaction Between Alexa-Herceptin-SH and Maleimide

0.75 mL of the Alexa-Herceptin-SH solution obtained in the foregoing (1.5 mg in terms of Herceptin) and 4 mL of the 10 mg/mL mixed polymer micelle solution were mixed using a CRYOVIAL®, and subjected to a reaction at 30° C. for 2 hours (addition reaction between maleimide in Maleimide-PEG-PBLG and SH-modified Herceptin). After the reaction, the resultant was stored at below 4° C. overnight, and then a particle diameter measurement was performed. In order to remove unreacted Alexa-Herceptin-SH contained in the reaction liquid, the resultant was concentrated 5-fold three times at below 4° C. and 3,000 rpm using a centrifugal ultrafiltration filter (manufactured by Merck Millipore Corporation, trade name: “AMICON® Ultra ULTRACEL®-100K: 100,000 MWCO”). To the supernatant after the centrifugal ultrafiltration, 5 equivalents of a 10 mg/mL L-cysteine PBS solution was added with respect to the stoichiometric amount of maleimide, and the mixture was subjected to reaction at room temperature for 30 minutes to quench the unreacted maleimide moiety in the micelle. In order to remove the excess of L-cysteine, the resultant was purified by gel filtration (manufactured by GE Healthcare Japan, trade name: “PD-10”). At this time, solution replacement was performed with a 10% sucrose aqueous solution (w/v) as the eluent to prevent micelle aggregation. The resultant antibody-bound micelle solution was sterilized by filtration (filter pore size: 0.22 μm) and subjected to a dispensing operation to complete the preparation of an Alexa 488-labeled Herceptin-bound micelle formulation. The particle diameter of the finished formulation was measured with a dynamic light scattering photometer. This micelle formulation corresponds to an aspect of the present teachings in which the content of the backbone polymer unit α is 0 mass %, the content of the backbone polymer unit β is 90 mass %, and the content of the backbone polymer unit γ is 10 mass %. It should be noted that this may be paraphrased as an aspect in which the content of a pseudo unit α having a hydrophilic group-carrying percentage in the hydrophobic segment chain of 0% is 40 mass % and the content of the unit β is 50 mass %.

### Test Example 2

An Alexa 488-labeled Herceptin-bound micelle formulation was prepared in the same manner as in Test Example 1 except that 10 mg of PEG-PBLG (OBn: 76.8%), 80 mg of PEG-PBLG (OBn: 100%), and 10 mg of Maleimide-PEG-PBLG (content: 1:8:1 in the stated order) were used. This micelle formulation corresponds to an aspect of the present teachings in which the content of the backbone polymer unit α is 10 mass % and the hydrophilic group-carrying percentage of the hydrophobic segment chain in the unit α is 23.2%. In addition, this micelle formulation, like Test Example 4 below, corresponds to an aspect of the present teachings in which the content of the backbone polymer unit β is 80 mass % and the content of the backbone polymer unit γ is 10 mass %.

### Test Example 3

An Alexa 488-labeled Herceptin-bound micelle formulation was prepared in the same manner as in Test Example 1 except that 40 mg of PEG-PBLG (OBn: 76.8%), 50 mg of PEG-PBLG (OBn: 100%), and 10 mg of Maleimide-PEG-PBLG (content: 4:5:1 in the stated order) were used. This micelle formulation corresponds to an aspect of the present teachings in which the content of the backbone polymer unit α is 40 mass % and the hydrophilic group-carrying percentage of the hydrophobic segment chain in the unit α is 23.2%. In addition, this micelle formulation, like Test Example 5 below, corresponds to an aspect of the present teachings in which the content of the backbone polymer unit β is 50 mass % and the content of the backbone polymer unit γ is 10 mass %.

### Test Example 4

An Alexa 488-labeled Herceptin-bound micelle formulation was prepared in the same manner as in Test Example 1 except that 10 mg of PEG-PBLG (OBn: 60.6%), 80 mg of PEG-PBLG (OBn: 100%), and 10 mg of Maleimide-PEG-PBLG (content: 1:8:1 in the stated order) were used. This micelle formulation corresponds to an aspect of the present teachings in which the content of the backbone polymer unit α is 10 mass % and the hydrophilic group-carrying percentage of the hydrophobic segment chain in the unit α is 39.4%.

### Test Example 5

An Alexa 488-labeled Herceptin-bound micelle formulation was prepared in the same manner as in Test Example 1 except that 40 mg of PEG-PBLG (OBn: 60.6%), 50 mg of PEG-PBLG (OBn: 100%), and 10 mg of Maleimide-PEG-PBLG (content: 4:5:1 in the stated order) were used. This micelle formulation corresponds to an aspect of the present teachings in which the content of the backbone polymer unit α is 40 mass % and the hydrophilic group-carrying percentage of the hydrophobic segment chain in the unit α is 39.4%.

### Test Example 6

An Alexa 488-labeled Herceptin-bound micelle formulation was prepared in the same manner as in Test Example 1 except that 20 mg of PEG-PBLG (OBn: 60.6%), 70 mg of PEG-PBLG (OBn: 100%), and 10 mg of Maleimide-PEG-PBLG (content: 2:7:1 in the stated order) were used. This micelle formulation corresponds to an aspect of the present teachings in which the content of the backbone polymer unit α is 20 mass % and the hydrophilic group-carrying percentage of the hydrophobic segment chain in the unit α is 39.4%. In addition, this micelle formulation corresponds to an aspect of the present teachings in which the content of the backbone polymer unit β is 70 mass % and the content of the backbone polymer unit γ is 10 mass %.

Cellular Uptake Test for Micelle Formulations

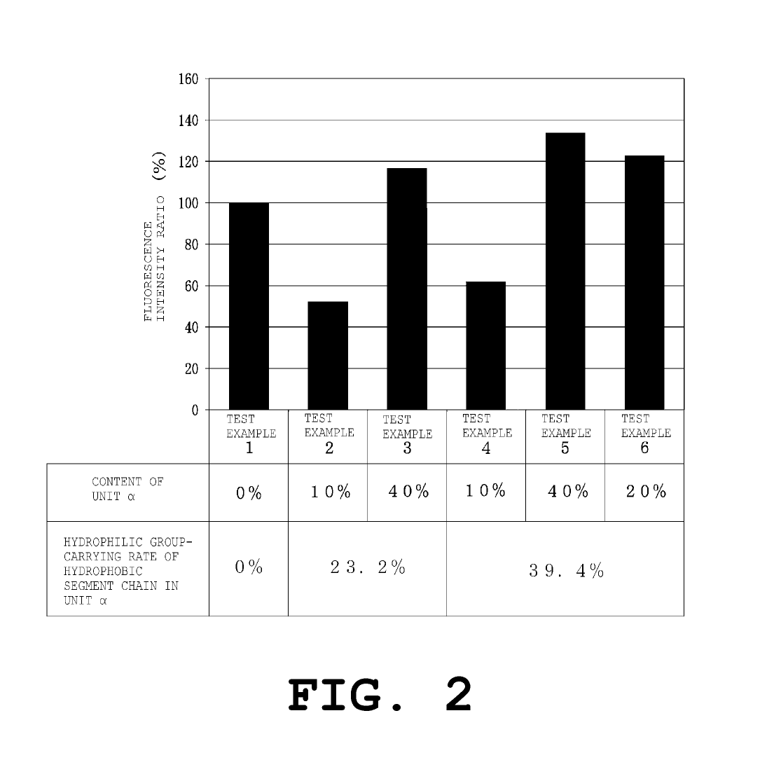

NCI-N87 cells were added in a state of being suspended in 90 μL of a medium so that about 250,000 of the cells were contained per 1.5-mL microtube. RPMI 1640 (Gibco®, Invitrogen) and 10% FBS (biowest) were used as the medium. The micelle formulations of Test Examples 1 to 6 were each added to a tube so as to achieve a final concentration of 4 μg/mL in terms of HERCEPTIN, and culturing was performed under a 5% CO₂ environment at 37° C. for 2 hours. The cells were washed with PBS twice, the cells were immobilized with 2% paraformaldehyde/PBS, and fluorescence intensity (FL1-A) was measured using a flow cytometer (manufactured by Nippon Becton Dickinson Company, Ltd., trade name: “BD Accuri® C₆ flow cytometer”). The resultant fluorescence intensity values were corrected to values with the polymer micelle concentration being adjusted to be constant, and the amount of each of the micelle formulations delivered to cell, more specifically, the total amount of the amount of the micelle formulation bound to the cell surface and the amount of the micelle formulation taken up into the cell, was evaluated. The results are shown in FIG. 2. It should be noted that the vertical axis of the graph of FIG. 2 is a relative value when the median value of the fluorescence intensity calculated in Test Example 1 is defined as 100%. As shown in FIG. 2, when the degree of hydrophilicity of the hydrophobic structure moiety in the micelle formulation resulting from the unit α is controlled (set) to a predetermined range, the delivery properties of the micelle formulation to the target cell is more certainly enhanced.

### INDUSTRIAL APPLICABILITY

The present teachings can be suitably utilized in the field of, for example, pharmaceutical formulations, such as anticancer agents.

1.-11. (canceled)

12. A polymer micelle pharmaceutical composition, comprising:
a block copolymer unit α having a hydrophilic polymer chain segment and a hydrophobic polymer chain segment;
a block copolymer unit β having a hydrophilic polymer chain segment and a hydrophobic polymer chain segment; and
a block copolymer unit γ having a hydrophilic polymer chain segment, which has a target binding site bound thereto, and a hydrophobic polymer chain segment;
wherein:
the block copolymer unit β has one or more copies of a drug bound thereto;
the block copolymer unit α is free of a target binding site and a drug;
the block copolymer unit α, the block copolymer unit β, and the block copolymer unit γ are arranged such that the hydrophilic polymer chain segments are directed radially outward and the hydrophobic polymer chain segments are directed radially inward;
the hydrophobic polymer chain segment of the block copolymer unit α is constituted of repeating units having side chains, at least one of the side chains having a hydrophilic group; and
the block copolymer unit α constitutes 15 wt % or more of the total weight of the polymer micelle pharmaceutical composition.

13. The polymer micelle pharmaceutical composition according to claim 12, wherein 20% to 80% of the side chains of the hydrophobic polymer chain segment of the block copolymer unit α have a hydrophilic group.

14. The polymer micelle pharmaceutical composition according to claim 12, wherein the block copolymer unit α constitutes 80 wt % or less of the total weight of the polymer micelle pharmaceutical composition.

15. The polymer micelle pharmaceutical composition according to claim 12, wherein the hydrophilic polymer chain segment of each of the block copolymer units α and α comprises a polyethylene glycol chain, and the hydrophobic polymer chain segment of each of the block copolymer units α and α comprises a polyamino acid chain.

16. The polymer micelle pharmaceutical composition according to claim 12, wherein the block copolymer unit α has a lower hydrophobicity than the block copolymer unit β owing to the hydrophilic group(s) of the side chains of the hydrophobic polymer chain segment.

17. The polymer micelle pharmaceutical composition according to claim 12, wherein:
the hydrophobic polymer chain segment of the block copolymer unit β is constituted of repeating units having side chains; and
the side chains of the hydrophobic polymer chain segment of the block copolymer unit α contain a greater number of hydrophilic groups than the side chains of the hydrophobic polymer chain segment of the block copolymer unit β.

18. The polymer micelle pharmaceutical composition according to claim 12, wherein the block copolymer unit β has a higher hydrophobicity than the block copolymer unit α owing to the drug.

19. The polymer micelle pharmaceutical composition according to claim 12, wherein the block copolymer unit β contains more hydrophilic groups than the block copolymer unit α, but has a higher hydrophobicity than the block copolymer unit α owing to the drug.

20. The polymer micelle pharmaceutical composition according to claim 13, wherein:
the block copolymer unit α constitutes 15-80 wt % of the total weight of the polymer micelle pharmaceutical composition;
the hydrophilic polymer chain segment of each of the block copolymer units α, β and γ comprises a polyethylene glycol chain, and the hydrophobic polymer chain segment of each of the block copolymer units α, β and γ comprises a polyamino acid chain;
the hydrophobic polymer chain segment of the block copolymer unit β is constituted of repeating units having side chains; and
the side chains of the hydrophobic polymer chain segment of the block copolymer unit α contain a greater number of hydrophilic groups than the side chains of the hydrophobic polymer chain segment of the block copolymer unit β.

21. A polymer micelle encapsulating a drug, comprising:
a block copolymer α having the formula A₁-B₁;
a block copolymer β having the formula A₂-B₂(-D); and
a block copolymer γ having the formula Z-A₃-B₃;
wherein:
A₁, A₂ and A₃ are each independently a hydrophilic polymer segment,
B₁, B₂ and B₃ are each independently a hydrophobic polymer segment,
D is the drug, and
Z is a target binding site,
with the provisos that:
one or more copies of the drug is (are) respectively bound to one or more side chains of the hydrophobic polymer segment of the polymer β,
at least one side chain of the hydrophobic polymer segment of the block copolymer α is a hydrophilic group and
the block copolymer α constitutes 15 wt % or more of the total weight of the polymer micelle.

22. The polymer micelle according to claim 21, wherein:
each of A₁, A₂ and A₃ comprises a polyethylene glycol; and
each of B₁, B₂ and B₃ comprises a polyamino acid.

23. The polymer micelle according to claim 22, wherein 20% to 80% of the side chains of the polyamino acid chain of the block copolymer β have a hydrophilic group.

24. The polymer micelle according to claim 22, wherein the polyamino acid is selected from the group consisting of:
polyglutamic acid, esters and amides thereof; and
polyaspartic acid, esters and amides thereof.

25. The polymer micelle according to claim 21, wherein the block copolymer α has one of the following formulae (I) or (II): 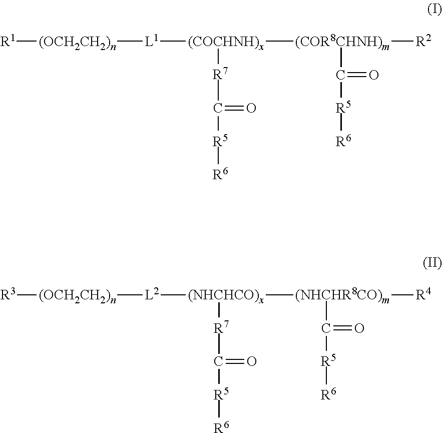
wherein:
R¹ and R³ are each independently a hydrogen atom or a lower alkyl group, which is unsubstituted or is substituted with an optionally protected functional group;
R² is a hydrogen atom, a saturated or unsaturated C₁ to C₂₉ aliphatic carbonyl group, or an arylcarbonyl group;
R⁴ is a hydroxy group, a saturated or unsaturated C₁ to C₃₀ aliphatic oxy group, or an aryl-lower alkyloxy group;
each R⁵ is independently —O— or —NH—;
10% to 90% of the total number (m+x) of R⁶ groups is/are (a) hydrogen atom(s), and the remaining R⁶ group(s) is/are (a) hydrophobic organic group(s);
R⁷ and R⁸ are each independently a methylene group or an ethylene group;
n is an integer from 10 to 2,500;
x is an integer from 10 to 300;
m is an integer from 0 to 300, with the proviso that, when m is 1 or more, the binding order of the repeating units, in which the number of repetitions is x and the repeating units in which the number of repetitions is m, is arbitrary, and the R⁶ groups are each independently selected in each repeating unit in one block copolymer;
L¹ is a linking group selected from —NH—, —O—, —O—Z—NH—, —CO—, —CH₂—, —O—Z—S—Z—, and —OCO—Z—NH—;
L² is a linking group selected from —OCO—Z—CO— and —NHCO—Z—CO—; and
each Z is independently a C₁ to C₆ alkylene group.

26. The polymer micelle according to claim 25, wherein 20% to 60% of the total number (m+x) of R⁶ groups are hydrogen atoms.

27. The polymer micelle according to claim 26, wherein the hydrophobic organic group(s) is (are) selected from the group consisting of a C₄ to C₁₆ alkyl group having a linear, branched, or cyclic structure, a C₆ to C₂₀ aryl group, a C₇ to C₂₀ aralkyl group and a sterol residue.

28. The polymer micelle according to claim 26, wherein the block copolymer α has a lower hydrophobicity than the block copolymer β.

29. The polymer micelle according to claim 22, wherein:
the polyamino acid of each of the block copolymer α, block copolymer β and block copolymer γ contains an average of 40 glutamic acids,
60% of the hydrogen atoms of the carboxylic acids in the side chains of the polyglutamic acid of the block copolymer α are substituted with phenyl groups,
the polyethylene glycol of each of the block copolymer α, block copolymer β and block copolymer γ has an average molecular weight of 10,000 Da,
D is docetaxel;
Z is a trastuzumab residue; and
the polymer micelle contains the block copolymer α, block copolymer β and block copolymer γ in a weight ratio of 4:5:1.

30. A method of treating cancer, comprising:
administering a therapeutically effective amount of the polymer micelle according to claim 29 to a patient in need thereof.

31. A method of treating cancer, comprising:
administering a therapeutically effective amount of the polymer micelle according to claim 21 to a patient in need thereof.
